(19) United States
(12) Patent Application Publication
(10) Pub. No.: US 2025/0154012 A1
(43) Pub. Date: May 15, 2025

(54) CO2 CONVERTING PROCESS USING PISTON REACTOR

(71) Applicant: Qatar Foundation for Education, Science and Community Development, Doha (QA)

(72) Inventors: Ma'moun Al-Rawashdeh, Doha (QA); Aya Emhemed Abousrafa, Doha (QA); Patrick Linke, Doha (QA); Kai Fischer, Doha (QA)

(21) Appl. No.: 18/940,328

(22) Filed: Nov. 7, 2024

Related U.S. Application Data
(60) Provisional application No. 63/548,252, filed on Nov. 13, 2023.

(51) Int. Cl. C01B 32/40; C07C 1/12

(52) U.S. Cl. CPC C01B 32/40; C07C 1/12

(57) ABSTRACT

A method of processing a feedstock is provided. The method includes processing the feedstock including carbon dioxide and hydrogen by utilizing an electro-mechanical device to process the feedstock. The processing of feedstock can occur via reverse water gas shift reaction and/or a methanation reaction.

### CROSS-REFERENCE TO RELATED APPLICATION

The present disclosure claims priority to U.S. Provisional Patent Application 63/548,252 having a filing date of Nov. 13, 2023, the entirety of which is incorporated herein.

### BACKGROUND

As many industrial plants, power plants, and marine and ship applications have or are planning to install CO₂ capture units, regulations related to CO₂ emissions are becoming stricter. Thus, while an abundant amount of CO₂ is expected to be available, more obstacles are expected to arise. Utilizing the CO₂ stream back into the process as fuels are central to reaching net zero emissions (or circularity), and/or to be used as a feedstock to make new chemical products to achieve a CO₂-negative technology.

An optimized CO₂ utilization and conversion process must be a very efficient and economical process and should (1) not generate CO₂, (2) be powered by renewable electricity (which can fluctuate as well as the price of electricity which is a central component in this process), and (3) activate the CO₂ molecule into a more reactive intermediate that is easier to process toward the target product. Hydrogen can activate the CO₂ molecule with the help of a catalyst. The CO₂ can be converted into a more reactive intermediate as synthesis gas (CO and H₂) that is used to make many valuable chemicals (e.g. methanol, liquid fuels), or to make CH₄ which is a fuel and also a valuable feedstock for many processes. A source of H₂ to react with CO₂ is electrolysis which is also powered by electricity. This technology is modular. Also, the source of CO₂ emissions is widely distributed. Thus, having a modularized CO₂ converting technology is desired.

There are several chemical reaction routes to convert CO₂ with H₂ into CO and/or CH₄ via reverse water gas shift (“RWGS”) and methanation. These reactions are widely used in the industry today via thermo-catalytic technologies. Heterogeneous catalyst is used that operated at high temperatures reached by burning fuels. This is undesired from CO₂ emissions standpoint. Also, these current technologies are not standalone processes. They are typically part of a large-scale process that has already these molecules. This means they take full advantage of the economy of scale, heat integration options, and available separation units and utilities. Also in these technologies, the aim is normally to better optimize the overall process and achieve the target purity which is not directly related to utilizing the CO₂. Because of this, current technology is not directly applicable for modular and or standalone CO₂ conversion process where the main aim is to fully utilize the CO₂ feedstock.

There are several technological development efforts that aim to develop modular and or stand-alone CO₂ conversion process that is powered by renewable electricity. Most of these efforts rely on electrochemical, electrothermal, and/or photochemical routes. Each of these routes has its advantages and limitations. Many of these options are still in the development stage. True assessments of all these technology options still need to be proven.

Additionally, piston reactors have been used with gases, such as, CH₄, CO, H₂, CO₂, and H₂O. However, these uses tend to be exothermic reactions that are partial combustion.

Therefore, a need exists for modularized CO₂ converting technology.

Additionally, a need exists for a CO₂ converting process that utilizes a piston reactor.

### SUMMARY

The present disclosure is generally related to a method of processing a feedstock including carbon dioxide and hydrogen comprises utilizing an electro-mechanical device to process the feedstock. A feature of the present technology is the dynamic operation which is well-established in engines and has a dynamic process which responds to electricity prices. Another feature of the present technology is there is no more efficient electrical conversion than to rotational mechanical movement as evident by many electric motors when compared to resistive or conductive heating.

In light of the disclosure herein and without limiting the disclosure in any way, in an aspect of the present disclosure, which may be combined with any other aspect listed herein unless specified otherwise, a method of processing a feedstock including CO₂ and H₂ comprises utilizing an electro-mechanical device to process the feedstock; and converting the feedstock into a product mixture including CO and H₂O via a reverse water gas shift reaction.

In another aspect of the present disclosure, which may be combined with any other aspect listed herein unless specified otherwise, the electro-mechanical device is a piston reactor.

In another aspect of the present disclosure, which may be combined with any other aspect listed herein unless specified otherwise, the product mixture further includes CH₄.

In another aspect of the present disclosure, which may be combined with any other aspect listed herein unless specified otherwise, the reverse water gas shift reaction is an endothermic reaction.

In another aspect of the present disclosure, which may be combined with any other aspect listed herein unless specified otherwise, a high selectivity of CO is produced.

In another aspect of the present disclosure, which may be combined with any other aspect listed herein unless specified otherwise, the method further comprises utilizing a high temperature preheating in the range of atmospheric temperature up to 600° C. or heterogeneous catalyst or homogenous catalyst.

In another aspect of the present disclosure, which may be combined with any other aspect listed herein unless specified otherwise, the method further comprises utilizing a co-feeding trigger.

In another aspect of the present disclosure, which may be combined with any other aspect listed herein unless specified otherwise, the co-feeding trigger includes oxygen and either ozone, spark plug, liquified Petroleum gas (LPG), or liquid hydrocarbon (C4-C8).

In another aspect of the present disclosure, which may be combined with any other aspect listed herein unless specified otherwise, a method of processing a feedstock including CO₂ and H₂ comprises utilizing an electro-mechanical device to process the feedstock; and converting the feedstock into a product mixture including CO and H₂O via a methanation reaction.

In another aspect of the present disclosure, which may be combined with any other aspect listed herein unless specified otherwise, the electro-mechanical device is a piston reactor.

In another aspect of the present disclosure, which may be combined with any other aspect listed herein unless specified otherwise, the product mixture further includes CO.

In another aspect of the present disclosure, which may be combined with any other aspect listed herein unless specified otherwise, the product mixture further includes CH₄, a high selectivity of CH₄ is produced.

In another aspect of the present disclosure, which may be combined with any other aspect listed herein unless specified otherwise, the method further comprises utilizing temperature preheating and heterogeneous or homogenous catalyst.

In another aspect of the present disclosure, which may be combined with any other aspect listed herein unless specified otherwise, the method further comprises utilizing a co-feeding trigger.

In another aspect of the present disclosure, which may be combined with any other aspect listed herein unless specified otherwise, the co-feeding trigger includes oxygen and either ozone, spark plug, liquified Petroleum gas (LPG), or liquid hydrocarbon (C4-C8).

In another aspect of the present disclosure, which may be combined with any other aspect listed herein unless specified otherwise, a method of processing a feedstock including CO₂ and H₂ comprises utilizing an electro-mechanical device to process the feedstock; converting the feedstock into a product mixture including CO and H₂O via a reverse water gas shift reaction; and converting the feedstock into a product mixture including CO and H₂O via a methanation reaction.

In another aspect of the present disclosure, which may be combined with any other aspect listed herein unless specified otherwise, the electro-mechanical device is a piston reactor.

In another aspect of the present disclosure, which may be combined with any other aspect listed herein unless specified otherwise, the product mixture further includes CH₄.

In another aspect of the present disclosure, which may be combined with any other aspect listed herein unless specified otherwise, the method further comprises utilizing temperature preheating and heterogeneous or homogenous catalyst.

In another aspect of the present disclosure, which may be combined with any other aspect listed herein unless specified otherwise, the method further comprises utilizing a co-feeding trigger.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

The patent or application file contains at least one drawing executed in color. Copies of this patent or patent application publication with color drawing(s) will be provided by the office upon request and payment of the necessary fee.

Features and advantages of the present disclosure, including a process including processing a feedstock including carbon dioxide and hydrogen, described herein may be better understood by reference to the accompanying drawings in which:

FIG. 1 is a schematic of a RWGS process, according to an example embodiment of the present disclosure.

FIG. 2 is a schematic of a methanation process, according to an example embodiment of the present disclosure.

FIG. 3 is a schematic of an integrated RWGS and methanation process, according to an example embodiment of the present disclosure.

FIG. 4A is a graph of CO₂% conversion of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor, according to an example embodiment of the present disclosure.

FIG. 4B is a graph of CO₂% selectivity of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor, according to an example embodiment of the present disclosure.

FIG. 4C is a graph of CH₄% selectivity of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor, according to an example embodiment of the present disclosure.

FIG. 5A is a graph of (a) CO₂% conversion, (b) CO % selectivity, and (c) CH₄% selectivity of an RWGS reaction at intake pressure of 1 bar, temperature from 400 K to 1400 K, ratios of H₂ to CO₂ from 1:1 till 1:4, at 3000 RPM in a piston reactor, according to an example embodiment of the present disclosure.

FIG. 5B is a graph of (a) CO₂% conversion, (b) CO % selectivity, and (c) CH₄% selectivity of an RWGS reaction at intake pressure of 1 bar, temperature from 400 K to 700 K, using a trigger at a ratio of H₂ to CO₂ of 4:1, at 3000 RPM in a piston reactor, according to an example embodiment of the present disclosure.

FIG. 5C is a graph of experimental data for (a) CO₂% conversion and (b) CO % selectivity of an RWGS reaction at an intake of atmospheric temperature and pressure, 1300 RPM, and a ratio of H₂ to CO₂ 1.86 in a piston reactor with a trigger, according to an example embodiment of the present disclosure.

The reader will appreciate the foregoing details, as well as others, upon considering the following detailed description of certain non-limiting embodiments of the present disclosure.

### DETAILED DESCRIPTION

The present disclosure is generally related to a method of processing a feedstock including carbon dioxide and hydrogen comprising utilizing an electro-mechanical device to process the feedstock. This is a stand-alone process installed near a source of CO₂-captured stream. The present disclosure may then be utilized to convert this CO₂ back into, for example, CO, CH₄, and/or CH₃OH that can be reused as sustainable fuels. The unit operates using renewable electricity and can be dynamically operate when it is only economical. Alternatively, the present disclosure may be integrated with an existing plant that utilizes synthesis gas with a desire to incorporate a more CO₂ sustainable process, for example, methanol or Fischer-Tropsch plants.

This present disclosure introduces a novel and unique approach to piston reactor technology which is an electro-mechanical-chemical-powered conversion device. This reactor is similar to a typical automotive internal combustion engine, but it is modified to be integrated with an electrical motor to drive endothermic reactions which is the opposite operation of current engines in which exothermic reactions drive the engine. The feedstock is not liquid or gas fuel to be combusted but instead a chemical feedstock as CO₂ or H₂, and the desired product is obtained from the exhaust, which is typically undesired in engines. The rapid adiabatic compressions and expansions result in an operating window that is unmet by many reactor technologies of very high temperatures and pressures for a short period. The very high temperature and pressure activate the reaction, and the rapid expansion may preserve the intermediate desired product. This could be beneficial for operating with reversible reactions such as RWGS and methanation. For these reactions, a catalyst or trigger is needed, as these reactions are not feasible in gas phase reactions on their own.

The electro-mechanical device “piston reactor” converts CO₂ feedstock with H₂ into either (1) CO and H₂O product mixture via RWGS, (2) CH₄ and H₂O via methanation, or (3) a combination of RWGS and methanation depending on conditions used and type of reaction triggers. The reactor operates either using or generating electricity and can be responsively integrated with the electricity grid. A process of multiple devices facilitating these conversions, coupled by transferring shaftwork across devices, can convert CO₂ in either net power generating or net power using modes. The system can be dynamically operated as electricity user and producer while converting CO₂.

The reactor and integrated processes comprised thereof are suitable for dynamic operation of fast and quick shutdown or for switching between power using and generating modes, thus well-suited for integration with electricity networks fed by intermittent generation sources such as solar or wind power. The reactor is modular which makes it suitable for implementation in larger scale/centralized as well as smaller scale/decentralized CO₂ conversion units.

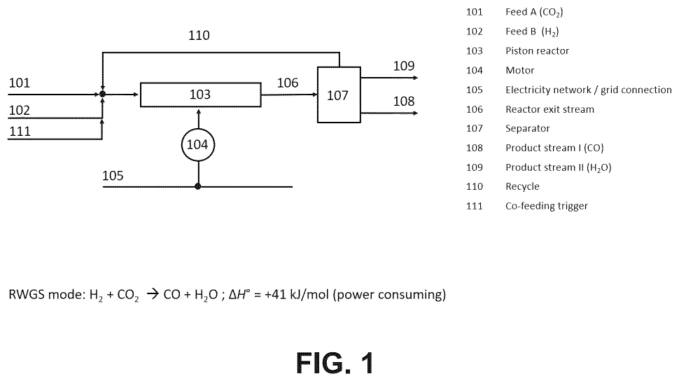

FIG. 1 illustrates a RWGS reaction (H₂+CO₂→CO+H₂O) having two feed streams of CO₂ 101 and H₂ 102. A co-feeding trigger stream 111 can be added to the feed streams of CO₂ 101 and H₂ 102. The feed streams 101, 102 (with or without the co-feeding trigger steam 111) enter a piston reactor 103 which utilizes a motor 104 powered by an electricity network and grid connection 105. From the piston reactor 103, the reactor exit stream 106 enters into a separator 107. The separator 107 outputs three feeds: a first product stream of CO 108, a second product stream of H₂O 109, and a recycle stream 110. The recycle stream 110 is returned to the piston reactor 103 along with the fresh feed streams 101, 102. The reaction has a ΔH° of +41 kJ/mol so the reaction is power consuming.

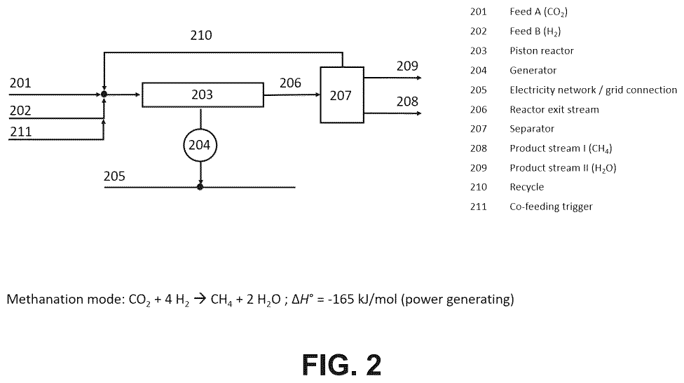

FIG. 2 illustrates a methanation reaction (4H₂+CO₂→CH₄+2H₂O) having two feed streams of CO₂ and H₂ 201, 202. A co-feeding trigger stream 211 can be added to the feed streams of CO₂ 201 and H₂ 202. The feed streams 201, 202 (with or without the co-feeding trigger steam 211) enter a piston reactor 203 which utilizes a generator 204 powered by an electricity network and grid connection 205. From the piston reactor 203, the reactor exit stream 206 enters into a separator 207. The separator 207 outputs three feeds: a first product stream of CH₄ 208, a second product stream of H₂O 209, and a recycle stream 210. The recycle stream 210 is returned to the piston reactor 203 along with the fresh feed streams 201, 202. The reaction has a ΔH° of −165 KJ/mol so the reaction is power generating.

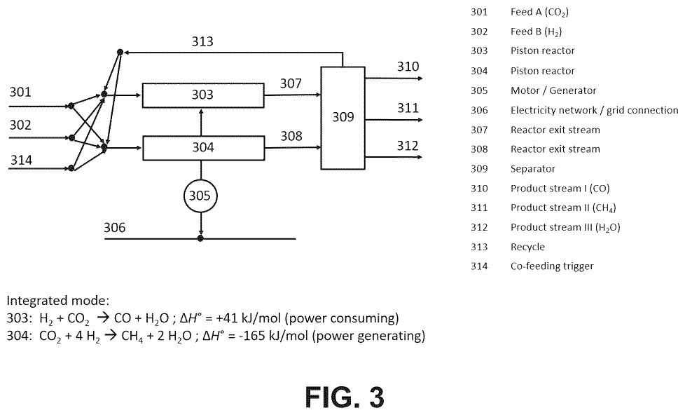

FIG. 3 illustrates an integrated process including both a RWGS reaction (H₂+CO₂→CO+H₂O) and a methanation reaction (4H₂+CO₂→CH₄+2H₂O) having two feed streams of CO₂ and H₂ 301, 302. A co-feeding trigger stream 314 can be added to the feed streams of CO₂ 301 and H₂ 302. The feed streams 301, 302 (with or without the co-feeding trigger steam 314) enter into two piston reactors 303, 304. The first piston reactor 303 initiates the RWGS reaction and the second piston reactor 304 initiates the methanation reaction. The second piston reactor 304 feeds a product into the first piston reactor 303. Additionally, the second piston reactor 304 utilizes a motor/generator 305 powered by an electricity network and grid connection 306. In some embodiments, the motor/generator 305 powers both the first piston reactor 303 and the second piston reactor 304. From the piston reactors 303, 304, the reactor exit streams 307, 308 enter into a separator 309. The separator 309 outputs four feeds: a first product stream of CO 310, a second product stream of CH₄ 311, a third product stream of H₂O 312, and a recycle stream 313. The recycle stream 313 is returned to the piston reactors 303, 304 along with the fresh feed streams 301, 302.

Certain features of the present disclosure are that the reactor operates using electricity and can be integrated with the electricity grid. The reactor is suitable for dynamic operation of fast and quick shutdown making it ideal for integration with renewable electricity. For example, the reactor operates only when the electricity price reaches a threshold. The reactor is modular which makes it ideal for decentralized production units. For the case of RWGS, the CO from the reactor can be reacted with H₂ from an electrolyzer or another available H₂ stream to make synthesis gas that can be used to make methanol, or Fischer-Tropsch products. For the case of methanation, this device can allow closing the loop when using combusting methane.

The performance of the RWGS and methanations reactions in the piston reactor have been simulated for a wide range of conditions and triggers using a well-established kinetic and piston reactor model. Types of co-feeding triggers that can enable these reactions in the piston reactor have been identified and may include ozone. The conditions and reaction effluent mix have been used to synthesize different process options to identify the most optimal and efficient one at the process level to convert CO₂ into synthesis gas and/or methane as a standalone process or integrated with an industrial plant that is dealing and processing with synthesis gas. Additionally, higher compression ratios can be used to result in higher in-cylinder peak temperatures. The higher compression ratios may be dependent on the design of the piston reactor, increased intake temperature, and use of low heat capacity diluent. Experimental tests were conducted at different conditions validating the claimed benefits.

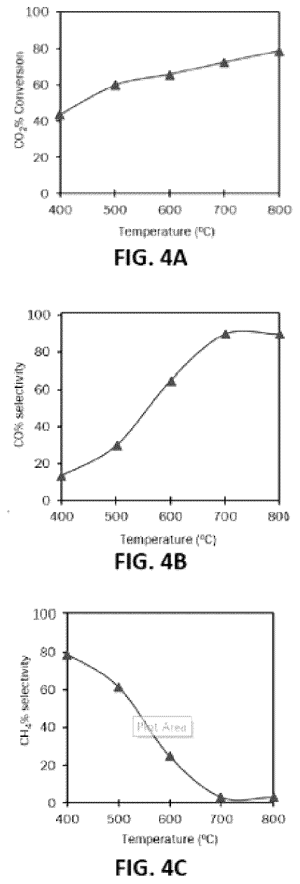

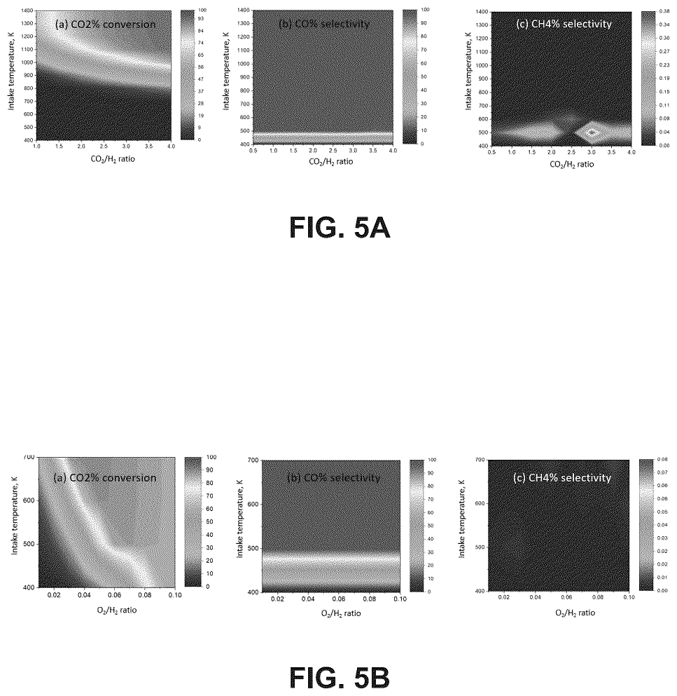

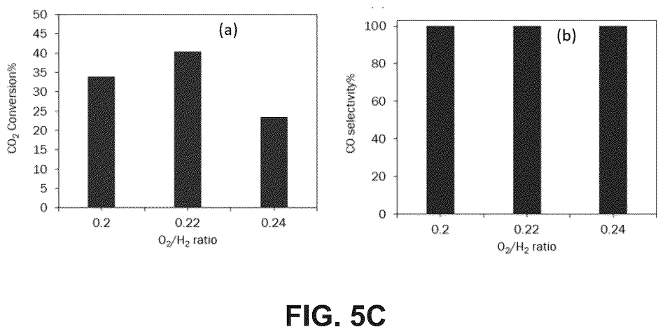

The present technology, in an embodiment, utilizes a RWGS reaction in a piston reactor, wherein the RWGS reaction is endothermic and is not a partial combustion reaction, and wherein the RWGS reaction has a high selectivity to CO with a high conversion to CO₂ when reacted with H₂ in a piston reactor. An example of the piston reactor utilizing the RWGS reaction is described below in further detail including with reference to FIGS. 4A-5C.

FIG. 4A is a graph of CO₂% conversion of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor. FIG. 4B is a graph of CO₂% selectivity of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor. FIG. 4C is a graph of CH₄% selectivity of an RWGS reaction at atmospheric pressure with a weight hourly space velocity of 12000 mL g_{cat}⁻¹ h⁻¹ and a ratio of H₂ to CO₂ of 4:1 in a conventional fixed-bed catalytic reactor.

FIGS. 4A to 5A (a) and 5B (a) illustrate the RWGS reaction performance for the conventional fixed-bed reactor versus the piston reactor, respectively. Both reactions are carried out at similar input conditions which is validated versus experimental data in FIG. 5C. However, in a conventional fixed-bed reactor, a heterogenous catalyst is used, whereas piston reactor does not use any heterogenous or homogenous catalysts, and provide much higher performance in terms of conversion, selectivity and at lower intake temperature conditions. In a conventional fixed-bed reactor, the CO₂ conversion, and selectivity to CO and CH₄ changes depending on the operating temperature. At low temperatures, selectivity to CH₄ is high as shown in FIGS. 4A to 4C. High selectivity to CO (above 80%) is only attainable at temperatures higher than 700° C. At this condition, the CO₂ conversion is around 70%.

Doing the same reaction in the piston reactor at different conditions is shown in FIG. 5. FIGS. 5A and 5B are model based results the RWGS reactions with and without triggers, respectively. FIG. 5C presents the experimental performance of RWGS in piston reactor for selected conditions.

FIG. 5A is a graph of (a) CO₂% conversion, (b) CO % selectivity and (c) CH₄% selectivity of an RWGS reaction at atmospheric pressure, intake temperature from 400 K to 1400 K, ratios of H₂ to CO₂ from 1:1 till 1:4, at 3000 RPM in a piston reactor using no heterogenous or homogenous catalysts. FIG. 5B is a graph of (a) CO₂% conversion, (b) CO % selectivity, and (c) CH₄% selectivity of an RWGS reaction at intake pressure of 1 bar, temperature from 400 K to 700 K, using a trigger at a ratio of H₂ to CO₂ of 4:1, at 3000 RPM in a piston reactor. FIG. 4C is a graph of experimental data for (a) CO₂% conversion % and (b) CO % selectivity of an RWGS reaction at atmospheric pressure, atmospheric intake temperature, 1300 RPM, and a ratio of H₂ to CO₂ 1.86 in a piston reactor using a trigger.

FIG. 5A illustrates the RWGS reaction performance for the piston reactor. The reaction is carried out using a mechanistic kinetic model which is validated versus experimental data. In the piston reactor, the CO₂ conversion, and selectivity to CO and CH₄ changes depending on the operating temperature and H₂ to CO ratio. The conversion ranges between 28% to 100%. High conversion (above 80%) is only attainable at temperatures above 727° C. At these conditions, the CO % selectivity is 100%, and CH₄% selectivity is below 0.04%.

FIG. 5B illustrates the RWGS reaction performance for a piston reactor using a trigger. The reaction is carried out using a mechanistic kinetic model which is validated versus experimental data. The piston reactor achieves CO₂% conversions above 80% at temperatures of 127° C. or higher. CO % selectivity is obtained 100% only when the temperature exceeds 200° C.

FIG. 5C illustrates experimentally obtained data for the RWGS reaction in the piston reactor using a trigger. The achieved CO₂% conversion ranges from 30% to 40% at ambient conditions (temperature of 25° C., pressure of 1 bar). For all the tested conditions the measured CO % selectivity is 100%.

Those model and experimental results prove the unique RWGS performance achieved by the piston reactor. Also, the piston reactor is operated using renewable electricity that drives the piston which is another advantage of this reactor technology. This means the fuel and its associated CO₂ generated from the combustion needed to drive this reaction are eliminated.

The results in FIGS. 4A to 5C and as further described above are just one example of the uniqueness of the piston reactor to operate the RWGS reaction. It is possible to further enhance this performance depending on the way the following elements, for example, are used and operated:
Heterogenous and homogenous catalyst: Type, location, and composition of the heterogeneous catalyst added inside the compression chamber of the piston. A catalyst can be placed in the location of the spark plug, or added as a coated layer on the inside of the piston chamber and the like. Homogenous catalyst can be co-feed along with the feeding streams.
Co-feeding triggers: Co-feeding molecules alongside the feedstock to be used as a reaction trigger, examples of these includes oxygen and radicals, such as ozone, spark plug or the like, or partial combustion of liquified Petroleum gas (LPG), or liquid hydrocarbon (C4-C8).
Higher in-cylinder peak temperatures: These could be achieved by a higher compression ratio which depends on the design of the piston reactor, increased intake temperature, use of low heat capacity diluent, and the like Argon.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method of processing a feedstock including CO₂ and H₂ comprising:
utilizing an electro-mechanical device to process the feedstock; and
converting the feedstock into a product mixture including CO and H₂O via a reverse water gas shift reaction.

2. The method according to claim 1, wherein the electro-mechanical device is a piston reactor.

3. The method according to claim 1, wherein the product mixture further includes CH₄.

4. The method according to claim 1, wherein the reverse water gas shift reaction is an endothermic reaction.

5. The method according to claim 1, wherein a high selectivity of CO is produced.

6. The method according to claim 1, further comprising utilizing a high temperature preheating in the range of atmospheric temperature up to 600° C. or heterogeneous catalyst or homogenous catalyst.

7. The method according to claim 1, further comprising utilizing a co-feeding trigger.

8. The method according to claim 7, wherein the co-feeding trigger includes oxygen and either ozone, spark plug, liquified Petroleum gas (LPG), or liquid hydrocarbon (C4-C8).

9. A method of processing a feedstock including CO₂ and H₂ comprising:
utilizing an electro-mechanical device to process the feedstock; and
converting the feedstock into a product mixture including CO and H₂O via a methanation reaction.

10. The method according to claim 9, wherein the electro-mechanical device is a piston reactor.

11. The method according to claim 9, wherein the product mixture further includes CO.

12. The method according to claim 9, wherein the product mixture further includes CH₄, wherein a high selectivity of CH₄ is produced.

13. The method according to claim 9, further comprising utilizing temperature preheating and heterogeneous or homogenous catalyst.

14. The method according to claim 9, further comprising utilizing a co-feeding trigger.

15. The method according to claim 14, wherein the co-feeding trigger includes oxygen and either ozone, spark plug, liquified Petroleum gas (LPG), or liquid hydrocarbon (C4-C8).

16. A method of processing a feedstock including CO₂ and H₂ comprising:
utilizing an electro-mechanical device to process the feedstock;
converting the feedstock into a product mixture including CO and H₂O via a reverse water gas shift reaction; and
converting the feedstock into a product mixture including CO and H₂O via a methanation reaction.

17. The method according to claim 16, wherein the electro-mechanical device is a piston reactor.

18. The method according to claim 16, wherein the product mixture further includes CH₄.

19. The method according to claim 16, further comprising utilizing temperature preheating and heterogeneous or homogenous catalyst.

20. The method according to claim 16, further comprising utilizing a co-feeding trigger.
